(19) United States
(12) Patent Application Publication
(10) Pub. No.: US 2025/0295450 A1
(43) Pub. Date: Sep. 25, 2025

(54) FLEXIBLE MULTI-ARM CATHETER WITH DIAMETRICALLY OPPOSED SENSING ELECTRODES

(71) Applicant: Biosense Webster (Israel) Ltd., Yokneam (IL)

(72) Inventors: Kevin Justin HERRERA, West Covina, CA (US); Christopher Thomas BEECKLER, Brea, CA (US); Joseph Thomas KEYES, Sierra Madre, CA (US); Assaf GOVARI, Haifa (IL)

(73) Assignee: Biosense Webster (Israel) Ltd., Yokneam (IL)

(21) Appl. No.: 19/233,248

(22) Filed: Jun. 10, 2025

Related U.S. Application Data
(63) Continuation of application No. 18/641,666, filed on Apr. 22, 2024, now Pat. No. 12,329,448.
Application No. 18/641,666 is a continuation of application No. 17/119,949, filed on Dec. 11, 2020, now Pat. No. 11,992,259.
Application No. 17/119,949 is a division of application No. 15/950,994, filed on Apr. 11, 2018, now abandoned.

(51) Int. Cl. A61B 18/14; A61B 5/00; A61B 18/00

(52) U.S. Cl. CPC A61B 18/1492; A61B 5/6858; A61B 2018/00375; A61B 2018/00577; A61B 2018/1467

(57) ABSTRACT

A medical instrument includes a shaft, multiple flexible spines and multiple electrodes. The shaft is configured for insertion into a body of a patient. The multiple flexible spines have respective first ends that are connected to a distal end of the shaft and respective second ends that are free-standing and unanchored. The spines are bent proximally such that the second ends are more proximal than the first ends. Each of the flexible spines includes a tensile layer configured to cause the flexible spine to bend proximally. The multiple electrodes are disposed over the flexible spines.

### CROSS REFERENCE TO RELATED APPLICATIONS

This application is a continuation of prior filed U.S. patent application Ser. No. 18/641,666 filed Apr. 22, 2024 (now U.S. Pat. No. 12,329,448, with Attorney Docket No.: BIO5904USCNT1-253757.000476), which is a continuation of prior filed U.S. patent application Ser. No. 17/119,949 filed Dec. 11, 2020 (now U.S. Pat. No. 11,992,259, with Attorney Docket No.: BIO5904USDIV1-253757.000266), which is a divisional of prior filed U.S. patent application Ser. No. 15/950,994 filed Apr. 11, 2018 (Attorney Docket No.: BIO5904USNP), the entire contents of which are hereby incorporated by reference as if set forth in full herein.

### FIELD OF THE INVENTION

The present invention relates generally to medical probes, and particularly to multi-electrode catheters.

### BACKGROUND

Various types of diagnostic-catheters and therapeutic-catheters may be used in cardiac diagnostic procedures. For example, U.S. Patent Application Publication 2016/0081746 describes a catheter adapted for mapping and/or ablation in the atria that has a basket-shaped electrode array with two or more location sensors with a deflectable expander. The catheter comprises a catheter body, a basket electrode assembly at a distal end of the catheter body, and a control handle at a proximal end of the catheter body. The basket electrode assembly has a plurality of electrode-carrying spines and an expander that is adapted for longitudinal movement relative to the catheter body for expanding and collapsing the assembly via a proximal end portion extending past the control handle that can be pushed or pulled by a user. The expander is also adapted for deflection in response to an actuator on the control handle that allows a user to control at least one puller wire extending through the catheter body and the expander.

As another example, U.S. Pat. No. 6,669,693 describes a device having a retractable and deployable umbrella body. The umbrella body includes ablation elements for circumferentially engaging and ablating a target tissue. The umbrella body is an adjustable, compliant cone-shaped member that may be deployed over a wide range of working diameters. The ablation elements are attached to spines and to a circumferential loop or loop segment attached to the spines. The ablation elements attached to the umbrella body can therefore conform to the geometry of the pulmonary vein ostium and provide circumferential contact, which permits more accurate ablation procedures.

International Patent Application Publication WO/2016/090175 (PCT/US2015/063807) describes in various embodiments, systems, devices and methods for modulating targeted nerve fibers (e.g., hepatic neuromodulation) or other tissue. The systems may be configured to access tortuous anatomy of or adjacent hepatic vasculature. The systems may be configured to target nerves surrounding (e.g., within adventitia of or within perivascular space of) an artery or other blood vessel, such as the common hepatic artery.

U.S. Patent Application Publication 2012/0172697 describes a medical device that has a flexible elongated body, a handle connected to the elongated body, at least one spine connected to the elongated body, and a flexible sheet attached to the at least one spine. The flexible sheet has a plurality of electrodes thereon, wherein the flexible sheet and the plurality of electrodes define a mapping assembly for mapping electrical information in tissue, and wherein the at least one spine and the flexible sheet is movable from a collapsed configuration to a deployed configuration.

### SUMMARY

An embodiment of the present invention provides a medical instrument including a shaft, multiple flexible spines and multiple electrodes. The shaft is configured for insertion into a body of a patient. The multiple flexible spines have respective first ends that are connected to a distal end of the shaft and respective second ends that are free-standing and unanchored. The spines are bent proximally such that the second ends are more proximal than the first ends. Each of the flexible spines includes a tensile layer configured to cause the flexible spine to bend proximally. The multiple electrodes are disposed over the flexible spines.

In some embodiments, the multiple electrodes are disposed over diametrically opposing surfaces of the flexible spines.

In some embodiments, the flexible spines and the electrodes include circuit board substrates, and metallic elements disposed on the circuit board substrates, respectively.

In some embodiments, the circuit board substrates are folded so that the multiple electrodes are disposed over diametrically opposing facets of the circuit board substrates.

In an embodiment, each of the tensile layers includes one or more tensile fibers configured to cause the flexible spine to bend proximally. In another embodiment, a tensile strength of the layer is greater than that of a Nitinol alloy layer of same dimensions. In one example, the tensile strength of the layer is greater than the ultimate tensile strength of fully annealed Nitinol (at approximately 895 MPa) and greater than the tensile strength of work hardened Nitinol (at approximately 1900 MPa).

There is additionally provided, in accordance with an embodiment of the present invention, a manufacturing method, including producing multiple flexible spines having multiple electrodes disposed thereon. The multiple flexible spines are mounted at a distal end of a shaft. The multiple flexible spines have respective first ends that are connected to a distal end of the shaft and respective second ends that are free-standing and unanchored, and the spines are bent proximally such that the second ends are more proximal than the first ends.

There is also provided, in accordance with an embodiment of the present invention, a manufacturing method, including patterning electrodes and conductive lines on multiple flexible circuit boards. Pairs of the flexible circuit boards are laminated with a layer of tensile material sandwiched between the circuit boards of each pair, so as to form flexible spines. The multiple flexible spines are mounted at a distal end of a shaft. The multiple flexible spines have respective first ends that are connected to a distal end of the shaft and respective second ends that are free-standing and unanchored, and the spines are bent proximally such that the second ends are more proximal than the first ends. A tensile layer included in each of the flexible spines is configured to cause the flexible spines to bend proximally.

There is further provided, in accordance with an embodiment of the present invention, a manufacturing method, including patterning electrodes and conductive lines on multiple flexible circuit boards. The flexible circuit boards are folded along respective longitudinal axes of the circuit boards, over one or more fibers made of a tensile material, such that the fibers become sandwiched between two diametrically opposing facets of the patterned flexible boards, so as to form flexible spines. The multiple flexible spines are mounted at a distal end of a shaft. The multiple flexible spines have respective first ends that are connected to a distal end of the shaft and respective second ends that are free-standing and unanchored, and the spines are bent proximally such that the second ends are more proximal than the first ends. A tensile layer included in each of the flexible spines is configured to cause the flexible spines to bend proximally.

The present invention will be more fully understood from the following detailed description of the embodiments thereof, taken together with the drawings in which:

### BRIEF DESCRIPTION OF THE DRAWINGS

FIG. 1 is a schematic, pictorial illustration of a catheter-based electro-anatomical mapping system, in accordance with an embodiment of the present invention;

FIG. 2 is a schematic, pictorial view of a soft multi-arm catheter, in accordance with an embodiment of the present invention;

FIGS. 3A-3D are schematic views that exemplify manufacturing stages of flexible spines comprising diametrically opposed electrodes, in accordance with some embodiments of the present invention; and

FIG. 4 is a schematic, detailed pictorial view of the soft multi-arm catheter of FIG. 2, in accordance with an embodiment of the present invention.

### DETAILED DESCRIPTION OF EMBODIMENTS

### Overview

Electrical signals in the myocardium may be recorded by sensing electrodes disposed on diagnostic cardiac catheters. Such sensing electrodes may be disposed at a catheter distal end comprising a rigid backbone structure, or over stiff arms. A rigid backbone structure may be made, for example, of spines that are secured at both their ends to a shaft, e.g., assembled into a basket catheter. Alternatively, arms that are stiff enough can be held at one end only.

In some cases, however, a rigid distal end may be problematic. For example, while mapping the myocardium to acquire electrical signals, diagnostic catheters made of rigid structures or stiff members can trigger ectopic heartbeats (i.e., irregular heart rhythm due to a premature heartbeat) by mechanical contact with the tissue. There is also a risk of perforation of the myocardium by, for example, the edge of a stiff spine.

Embodiments of the present invention that are described hereinafter provide a multi-arm catheter comprising multiple flexible spines, referred to hereinafter as a ‘flexible multi-arm catheter.’ The flexible spines comprise a high density of diametrically opposing sensing electrode-pairs. The multiple flexible spines extend outward from a center of the distal end of the shaft that the soft catheter is fitted at and then curve inward towards the shaft and backwards in the proximal direction over the distal end of the shaft. Each flexible spine bends without being anchored at its other end, e.g., by embedding tensile material in the spine.

In other words, a given flexible spine has a respective first end that is connected to the distal end of the shaft and a respective second end that is free-standing and unanchored, wherein the spine is bent proximally such that the second end is more proximal than the first end.

Some disclosed embodiments utilize a flexible circuit board to construct spines that have electrodes on both the top-side and under-side (i.e., over diametrically opposing surfaces of the flexible circuit board). Such a geometry enables measuring signals from two opposing directions per each electrode-pair location. When the flexible spines are fully extended, the electrodes that face the shaft will typically not be in contact with tissue. These electrodes may be clinically significant when the spines are still partially advanced out of the sheath, when these ‘interior’ electrodes are still on the exterior side, and sensing may commence while the catheter is still in a compact form, being partially folded in the sheath.

In some embodiments, the circuit board is made of a flexible material that allows the circuit board to be tightly folded, in order to form the electrodes on two opposing sides. A thin material with high tensile strength, such as Vectran® or Ultra High Molecular Weight Polyethylene (UHMWPE), can be sandwiched between the two facets of the folded circuit board to force the flexible circuit board to bend. The resulting spine geometry provides additional structural and clinical safety, by avoiding contact of sharp edges with the myocardium.

Alternatively, or additionally, the flexible spine may include one or more high-tensile-strength fibers to control its bending, such as ones made of Liquid Crystal Polymer (LCP), Carbon Fiber, Fiberglass, and/or UHMWPE. In some embodiments, the flexible circuit has the electrodes conductive lines patterned as thin films and/or as an embedded yarn, in a way that maintains the structural flexibility of the flexible spine.

In some embodiments, the tensile strength of the material used for forcing the flexible circuit board to bend is greater than that of Nitinol alloys. Namely, for a same layer or a same fiber-thickness, the tensile force exerted by the layer or a fiber using one of the above listed materials, is higher than if made of one of Nitinol alloys. An example of catheter arms that are made of a Nitinol alloy, are the arms of a Pentaray® sensing-catheter, made by Biosense Webster, Irvine, California.

The disclosed flexible multi-arm catheter, whose arms are self-bending proximally and inwardly while being suspended from the distal end of the shaft, can accommodate any anatomy with high flexibility and with minimum stiffness. This design allows the physician to safely maneuver the catheter within a cardiac chamber and collect signals from tissue with less risk of ectopic beats or perforation. The soft multi-arm catheter thus expands the capabilities of a physician to diagnose certain cardiac disorders, especially in patients who are more vulnerable to side-effects described above of cardiac catheterization. Moreover, the flexible multi-arm design can increase the accessibility to mapping of anatomy parts hard to access with existing designs.

### System Description

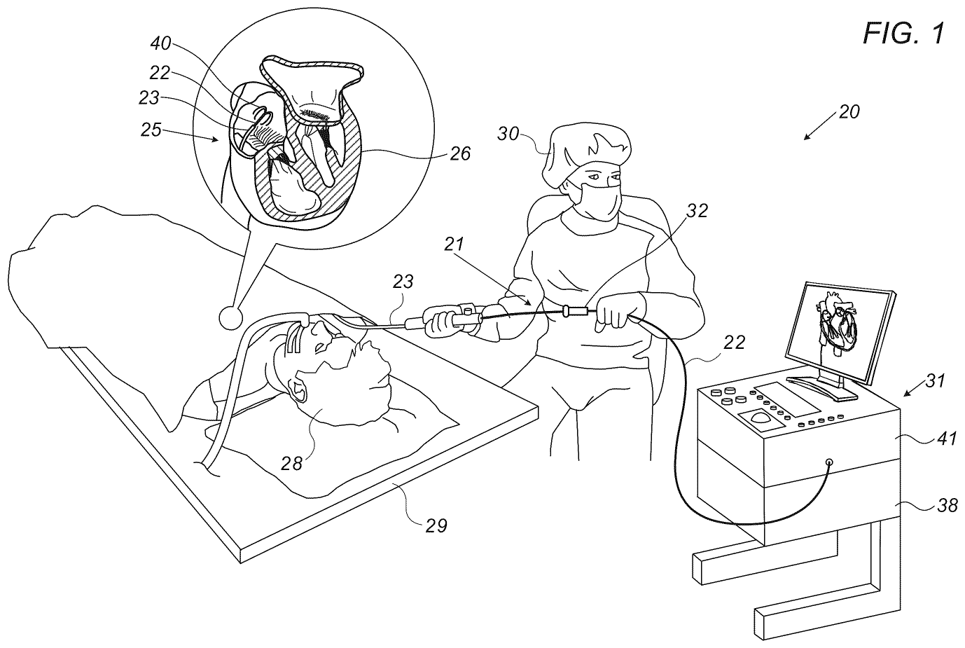

FIG. 1 is a schematic, pictorial illustration of a catheter-based electro-anatomical mapping system 20, in accordance with an embodiment of the present invention. System 20 comprises a catheter 21, wherein a shaft 22 of the catheter is inserted into a heart 26 of a patient 28 through a sheath 23. The proximal end of catheter 21 is connected to a control console 31. In the embodiment described herein, catheter 21 may be used for any suitable diagnostic purposes, such as electrophysiological mapping and/or electro-anatomical mapping of tissue in heart 26.

Console 31 comprises a processor 38, typically a general-purpose computer, with suitable front end. Console 31 comprises also an interface circuitry 41 for receiving signals from catheter 21, as well as for connecting to other components of system 20 that processor 38 controls.

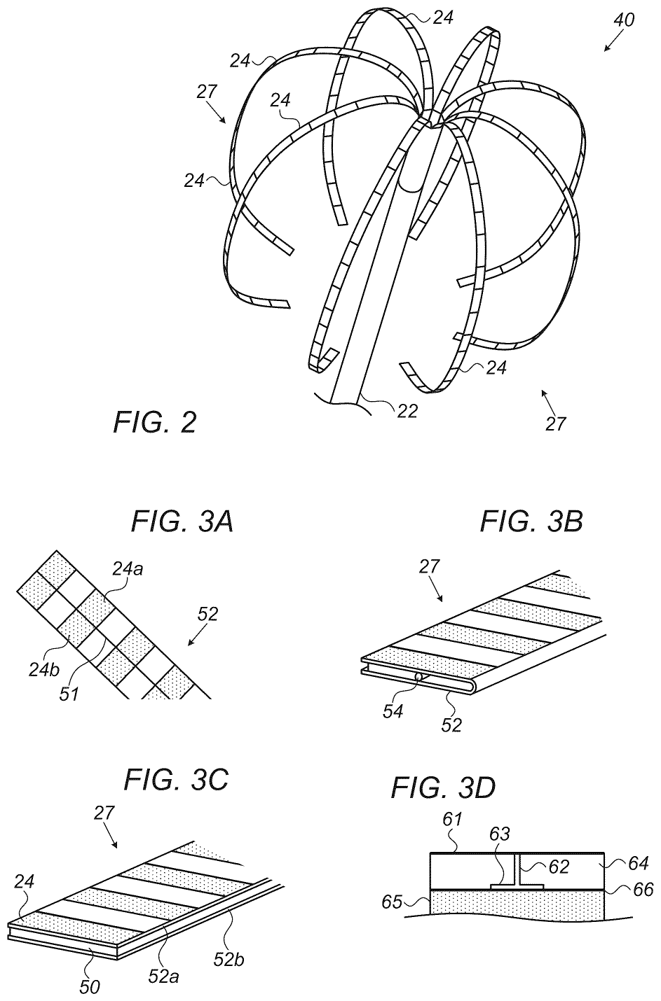

A physician 30 inserts shaft 22 through the vascular system of patient 28 lying on a table 29. As seen in an inset 25, catheter 21 comprises a soft multi-arm sensing catheter 40 fitted at the distal end of shaft 22 (after being advanced outside sheath 23). During the insertion of shaft 22, soft multi-arm catheter 40 is maintained in a collapsed configuration by sheath 23. By containing catheter 40 in a collapsed configuration, sheath 23 also serves to minimize vascular trauma along the way to target location. Physician 30 navigates soft multi-arm catheter 40 to a target location in heart 26 by manipulating shaft 22 using a manipulator 32 near the proximal end of the catheter and/or deflection from the sheath 23. Once the distal end of shaft 22 has reached the target location, physician 30 retracts sheath 23, or advances shaft 22, letting soft multi-arm sensing catheter 40 expand. The physician then operates console 31 so as sense signals using electrodes 24 (seen in FIG. 2) from tissue at the target location.

Although the pictured embodiment relates specifically to the use of a soft multi-arm sensing catheter 40 for electrophysiological sensing of heart tissue, the elements of system 20 and the methods described herein may additionally be applied in controlling multi-electrode ablation devices, such as circular ablation catheters, balloon ablation catheters, and multi-arm ablation devices.

Soft Multi-Arm Catheter with Diametrically Opposed Sensing Electrodes

FIG. 2 is a schematic, pictorial view of soft multi-arm sensing catheter 40, in accordance with an embodiment of the present invention. As seen, soft multi-arm catheter 40 is fitted on a distal end of shaft 22. Catheter 40 is made of multiple flexible spines that extend diagonally outward from a center of the distal end of shaft 22. The spines then bend inwardly, in the proximal direction, over the distal end of shaft 22, with their other end free-standing and unanchored. A multiplicity of metallic elements in the form of rectangular sensing electrodes 24 are patterned over the two facets of flexible spines 27 so as to allow detection of signals from opposing directions.

Electrodes 24 that are facing the shaft after the spines fully expand may still be clinically significant prior to spines 27 being fully advanced out of the sheath. When spines 27 are partially advanced out, such interior electrodes are on the exterior side, and sensing may commence while the catheter is still in a compact form, being partially folded in the sheath.

Flexible spines 27 are practically semi-floating so as to gently accommodate an anatomy that the spines may come in contact with. The edges of spines 27 are pointing toward shaft 22 so as to avoid sharp contact of an edge of a spine with tissue.

Spines 27 are designed to apply elastic opposing force when pressed inward, for example when pressed against a surface of tissue. The strength of the elastic opposing force can be tuned during design and/or manufacturing, so as to optimize the flexibility of catheter 40. In an embodiment, the opposing elastic force is made strong enough to ensure firm contact of electrodes 24 with tissue, but still weak enough to minimize undesired events such as ectopic heartbeats upon mechanical contact of one or more spines 27 of catheter 40 with myocardium tissue.

The example illustration shown in FIG. 2 is chosen purely for the sake of conceptual clarity. Other configurations of flexible spines are possible. Alternative or additional patterns are possible, such as circular electrodes, as well as fitting additional types of patterned sensors or electrodes, e.g., ablative, strain, ultrasound, or any other suitable type of sensor or electrode. The cross-section of flexible spines 27 may vary in shape. The distribution and number of electrodes that may encompass the flexible spines may vary. For example, ring shaped electrodes may be disposed over flexible spines having a circular cross-section.

FIGS. 3A-3D are schematic views that exemplify manufacturing stages of flexible spines 27 comprising diametrically opposed electrodes, in accordance with some embodiments of the present invention. In general, the flexible spines and the electrodes comprise circuit board substrates, and metallic elements disposed on the circuit board substrates, respectively.

FIG. 3A shows a flexible circuit board 52 before being folded to form a spine 27. Sensing electrodes 24a and 24b are patterned over circuit board 52 with a folding line 51 separating them physically and electrically. Once flexible circuit board 52 will be folded along folding line 51, electrodes 24a and 24b will form the diametrically opposing electrodes geometry, as further described below.

FIG. 3B shows a folded spine 27, which was made by folding circuit board 52 to achieve the diametrically opposing electrodes disposed on spine 27. The folded circuit board 52 wraps one or more tensile fibers 54 that run along the interior of the spine and provide the required structural strength and the tendency of spine 27 to bend as it is fixed on one of its ends.

FIG. 3C shows another embodiment, in which flexible spine 27 is made of a layer of a tensile material 50, such as Liquid Crystal Polymer (LCP), Ultra High Molecular Weight Polyethylene (UHMWPE), para-aramid, carbon fiber, or glass fiber, which is laminated in between two flexible circuit boards 52a and 52b (that were made, for example, by cutting circuit board 52 into two along line 51). Sensing electrodes 24 are patterned over boards 52a and 52b to face opposite directions.

FIG. 3D provides a zoom-in cross-sectional view of a patterning scheme of electrode 24 on a flexible board 65. In an embodiment, electrode layer 61 is made of copper deposited on an insulating polyimide layer 64. Nickel is next deposited then onto the copper, and finally gold is deposited onto the nickel. In an alternative embodiment, electrode layer 61 is made of a Titanium Tungsten (TiW) seed layer sputtered onto insulating polyimide layer 64. Gold is next sputtered onto the seed layer, and then a final layer of gold is added in a plating process. A conductive trace 63 for electrode 24 is embedded underneath polyimide layer 64 so as to connect electrode 24 to system 20, wherein the polyimide insulating layer 64 isolates trace 63 from the electrode layer 61. In an embodiment, conductive trace 63 is made of copper encapsulated in a layer 66 of gold. A via 62 (a through hole) is formed (e.g., by etching or drilling through printed board 52) and plated with gold, so as to electrically connect electrode 24 with conductive trace 63. In an embodiment, via 62 extends deeper and all the way through flexible board 65, to connect the diametrically opposing electrodes (FIG. 3D shows only the one facet coated with an electrode, while for connecting the opposing electrode the via extends much deeper to reach the other electrode on the other facet of flexible board 65).

An adhesive layer 66 bonds polyimide layer 64 to flexible board 65 so as to provide additional endurance and aid in manufacturing the multiple layers.

The examples of manufacturing designs shown in FIGS. 3A-3D are chosen purely for the sake of conceptual clarity. In alternative embodiments, the patterned designs may include different number and types of electrodes. The processing technologies of the different parts and layers of flexible spines 27 may vary.

In an embodiment, a flexible multi-arm catheter is provided, having up to thirty-two sensing electrodes 24 (e.g., sixteen opposing pairs) patterned on each flexible spine 27. Cather 40 is made of eight spines, making the total number of sensing-electrodes disposed at catheter 40 up to 256 electrodes.

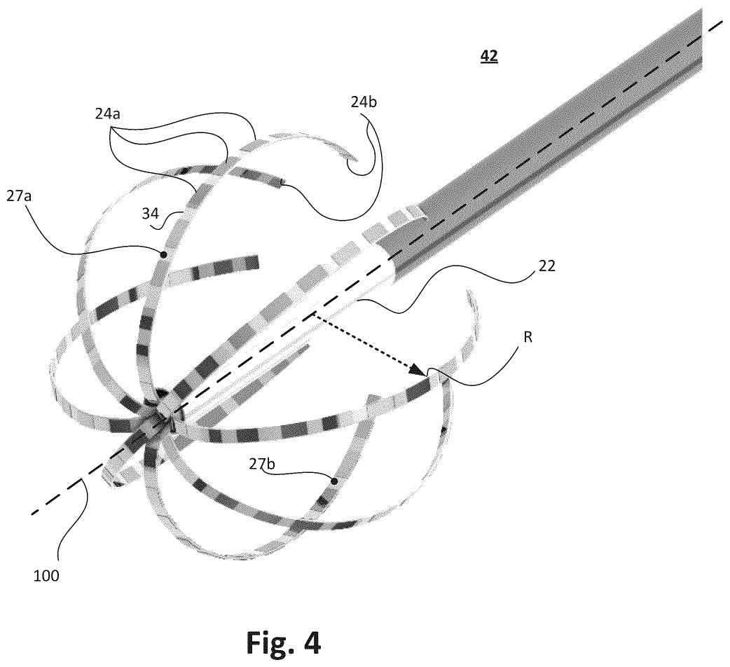

FIG. 4 is a schematic, detailed pictorial view of the soft multi-arm catheter of FIG. 2, in accordance with an embodiment of the present invention. For example, catheter 42 in FIG. 4 shows 12 electrode pairs 24 on each spine 27, each electrode pair 24 having an electrode 24a that faces away from a longitudinal axis 100 and an electrode 24b that faces the longitudinal axis 100. The spine 27 is configured such that when the spines 27 are fully extended from tubular shaft 22, a radius of curvature R can be conformed onto a portion of the interior surface 27b of the spine. As seen, spines 27 extend outward and then bend proximally and inwardly, relative to a longitudinal axis 100 of shaft 22. In an embodiment, tensile material 50 is configured to cause flexible spines 27 to bend freely at a preset radius of curvature R (i.e., the radius of the approximately circular arc shape that flexible spines 27 have) of about 0.40 inches with respect to a longitudinal axis 100, so as to have flexible spines 27 fit into a curved anatomy having similar typical size, such as of an ostium of a pulmonary vein.

In general, after catheter 40 is fully deployed, at least part of electrodes 24a will come in physical contact with tissue. Electrodes 24b, on the other hand, will typically not be in contact with tissue (rather, with blood only). Electrodes 24b may be clinically significant when the spines are still partially advanced out of the sheath, when these ‘interior’ electrodes are still on the exterior side, and sensing may commence while the catheter is still in a compact form, being partially folded in the sheath. Additionally, electrodes 24b may be used for the collection of background (e.g., far-field) electrophysiological signals, which processor 41 may utilize for the analysis of tissue electrophysiological signals from respective electrodes 24a.

In an embodiment, the size of electrodes 24a and 24b is both about 0.040 by about 0.027 inches, in width times length, respectively. The length of gap 34 between neighboring electrodes 24 is about 0.030 inches. The size of electrodes and gaps is designed such that it provides the medically required spatial resolution of intra-cardiac measured electrophysiological signals. The exemplary configurations described and illustrated herein allow for the elimination of a rigid backbone member such as a Nitinol wire in the spine while allowing detection of signals from both side of spine 27 via outer electrode 24a and inner electrode 24b (FIG. 4) for each electrode pair 24. This can be achieved by configuring each spline to have a flexible circuit substrate. The substrate has conductive surfaces on the outer surface of the substrate as electrodes. By using the thin flexible circuit with a suitable tensile member (e.g., polymeric fiber) disposed between the surfaces, a physician can maneuver the spines 27 in the heart and collect signals regardless of the orientation of the spines 27 as well as a lower risk of complications due to the spine members.

Although the embodiments described herein mainly address cardiac electrophysiological mapping and/or electroanatomical mapping, the methods and systems described herein can also be used in other applications, such as otolaryngology or neurology procedures.

It will thus be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A medical instrument, comprising:
a catheter shaft for insertion into a body of a patient, the catheter shaft extending along a longitudinal axis from a proximal end to a distal end;
a plurality of spines connected to the distal end of the catheter shaft, each spine comprising:
a first end that is connected to a distal end of the catheter shaft;
a second end that is free-standing and unanchored;
a first circuit board substrate;
a second circuit board substrate;
a tensile member disposed in between and spacing the first circuit board substrate and the second circuit board substrate;
a first plurality of electrodes disposed on a first facet of the first circuit board substrate; and
a second plurality of electrodes disposed on a second facet of the second circuit board substrate that is diametrically opposed to the first facet, the second plurality of electrodes being separated electrically and physically from the first plurality of electrodes.

2. The medical instrument of claim 1, wherein the first plurality of electrodes face the longitudinal axis and the second plurality of electrodes face away from the longitudinal axis.

3. The medical instrument of claim 1, wherein each spine is bent proximally such that the second end is more proximal than the first end.

4. The medical instrument of claim 3, wherein each spine has a radius of curvature of approximately 0.40 inches with respect to the longitudinal axis.

5. The medical instrument of claim 1, wherein each spine extends diagonally outward from a center of the distal end of the catheter shaft and bends inwardly, in a proximal direction, towards the longitudinal axis.

6. The medical instrument of claim 1, wherein the tensile member comprises a tensile layer, the tensile layer of each spine being configured to cause the respective spine to bend proximally.

7. The medical instrument according to claim 6, wherein the tensile layer comprises one or more tensile fibers.

8. The medical instrument according to claim 7, wherein a tensile strength of the tensile layer is greater than that of a Nitinol alloy layer of same dimensions.

9. The medical instrument of claim 8, wherein the tensile strength of the tensile layer is greater than 895 MPa.

10. The medical instrument of claim 1, wherein the tensile member comprises a layer of tensile material laminated between the diametrically opposing facets of the first and second circuit board substrates.

11. A manufacturing method, comprising:
patterning a plurality of first electrodes and a plurality of first conductive lines on a first flexible circuit board;
patterning a plurality of second electrodes and a plurality of second conductive lines on a second flexible circuit board, the second plurality of electrodes being separated electrically and physically from the first plurality of electrodes;
laminating pairs of the first and second flexible circuit boards, with a layer of tensile material sandwiched between the first and second circuit boards of each pair, so as to form a plurality of flexible spines; and
mounting the plurality of flexible spines at a distal end of a shaft, each flexible spine having a first end that is connected to a distal end of the shaft and a second end that is free-standing and unanchored.

12. The manufacturing method of claim 11, wherein each spine is bent proximally such that the second ends are more proximal than the first ends, and wherein the tensile material included in each of the flexible spines is configured to cause the flexible spines to bend proximally.

13. The manufacturing method of claim 11, wherein each spine has a radius of curvature of approximately 0.40 inches with respect to a longitudinal axis of the shaft.

14. The manufacturing method of claim 11, wherein a tensile strength of the tensile material is greater than 895 MPa.

15. The manufacturing method of claim 11, wherein the tensile layer comprises one or more tensile fibers.

16. A manufacturing method, comprising:
producing a plurality of flexible spines having a plurality of first electrodes and a plurality of second electrodes disposed thereon and diametrically opposing one another, the second plurality of electrodes being separated electrically and physically from the first plurality of electrodes; and
mounting the plurality of flexible spines at a distal end of a shaft, each flexible spine having a first end that is connected to a distal end of the shaft and a second end that is free-standing and unanchored, each flexible spine being bent proximally such that the second end is more proximal than the first end, and each flexible spine comprising a tensile material configured to cause the respective flexible spine to bend proximally.

17. The manufacturing method according to claim 16, wherein producing the plurality of flexible spines comprises, for each respective flexible spine, disposing the plurality of first electrodes on a first circuit board substrate and the plurality of second electrodes on a second circuit board substrate.

18. The manufacturing method of claim 17, wherein producing the plurality of flexible spines comprises, for each respective flexible spine, laminating the first and second flexible circuit board substrates, with the tensile material sandwiched between the first and second circuit board substrates.

19. The manufacturing method according to claim 16, wherein producing the plurality of flexible spines comprises fitting one or more tensile fibers in each of the plurality of flexible spines, so as to cause the flexible spine to bend proximally.

20. The manufacturing method according to claim 16, wherein producing the plurality of flexible spines comprises including a tensile layer with tensile strength that is greater than that of a Nitinol alloy layer of same dimensions.
